(19) United States
(12) Patent Application Publication
(10) Pub. No.: US 2024/0420480 A1
(43) Pub. Date: Dec. 19, 2024

(54) CONTROL DEVICE, CONTROL SYSTEM, AND CONTROL METHOD

(71) Applicant: Mitsubishi Electric Corporation, TOKYO (JP)

(72) Inventors: Kaori HANDA, Tokyo (JP); Yasunori TSUBAKI, Tokyo (JP); Kotoyu SASAYAMA, Tokyo (JP); Masaki HAMADA, Tokyo (JP)

(73) Assignee: Mitsubishi Electric Corporation, TOKYO (JP)

(21) Appl. No.: 18/819,455

(22) Filed: Aug. 29, 2024

Related U.S. Application Data
(63) Continuation of application No. PCT/JP2022/019009, filed on Apr. 27, 2022.

(51) Int. Cl. G06V 20/52; A61L 9/00; F24F 11/63; G06F 3/14; G06F 3/16; G06T 7/20; G06V 40/10; H05B 47/125

(52) U.S. Cl. CPC G06V 20/53; A61L 9/00; F24F 11/63; G06F 3/14; G06F 3/165; G06T 7/20; G06V 40/10; H05B 47/125; A61L 2209/111; G06T 2207/10016; G06T 2207/30196; G06T 2207/30232; G06T 2207/30242

(57) ABSTRACT

A control device controls an output device that outputs a stimulus. The control device includes an acquisition unit that acquires a video obtained by an image capturing device by capturing images of a plurality of people existed in a place where movement in a plurality of directions is possible and a control unit that detects a moving people count as a number of people who moved in a first direction among the plurality of directions based on the video, calculates a ratio of the people who moved in the first direction by using the moving people count, and when a situation where the ratio is less than a predetermined threshold value is continuing for a predetermined set time, executes control for changing the stimulus currently outputted by the output device to a different stimulus.

### CROSS-REFERENCE TO RELATED APPLICATION

This application is a continuation application of International Application No. PCT/JP2022/019009 having an international filing date of Apr. 27, 2022.

### BACKGROUND OF THE INVENTION

### 1. Field of the Invention

The present disclosure relates to a control device, a control system, and a control method.

### 2. Description of the Related Art

Displays are installed in train stations, shopping malls, and so forth. For example, information for guiding people is outputted to the display. A technology for outputting the information to the display is described in Patent Reference 1, for example.

Patent Reference 1: Japanese Patent Application Publication No. 2014-215662

Incidentally, a person receives a stimulus. For example, the stimulus can be a stimulus by visual sensation, a stimulus by auditory sensation, or the like. As people get accustomed to a stimulus, the people stop responding to the stimulus.

### SUMMARY OF THE INVENTION

An object of the present disclosure is to make people respond to a stimulus.

A control device according to an aspect of the present disclosure is provided. The control device controls an output device that outputs a stimulus. The control device includes an acquisition unit that acquires a video obtained by an image capturing device by capturing images of a plurality of people existed in a place where movement in a plurality of directions is possible and a control unit that detects a moving people count as a number of people who moved in a first direction among the plurality of directions based on the video, calculates a ratio of the people who moved in the first direction by using the moving people count, and when a situation where the ratio is less than a predetermined threshold value is continuing for a predetermined set time, executes control for changing the stimulus currently outputted by the output device to a different stimulus.

According to the present disclosure, it is possible to make people respond to a stimulus.

### BRIEF DESCRIPTION OF THE DRAWINGS

The present disclosure will become more fully understood from the detailed description given hereinbelow and the accompanying drawings which are given by way of illustration only, and thus are not limitative of the present disclosure, and wherein:

FIG. 1 is a diagram showing a control system in a first embodiment;

FIG. 2 is a diagram showing an installation example of a camera and an output device in the first embodiment;

FIG. 3 is a diagram showing the hardware included in a control device in the first embodiment;

FIG. 4 is a block diagram showing functions of the control device in the first embodiment;

FIG. 5 is a flowchart showing an example of a process executed by the control device in the first embodiment;

FIG. 6 is a diagram showing an example of a management table in a first modification of the first embodiment;

FIG. 7 is a flowchart showing an example of a process executed by a control device in the first modification of the first embodiment;

FIG. 8 is a diagram showing an example of a threshold value table in a second modification of the first embodiment;

FIG. 9 is a flowchart showing an example of a process executed by a control device in the second modification of the first embodiment;

FIG. 10 is a block diagram showing functions of a control device in a second embodiment;

FIG. 11 is a flowchart showing an example of a process executed by the control device in the second embodiment;

FIG. 12 is a flowchart showing an example of a process executed by a control device in a first modification of the second embodiment;

FIG. 13 is a diagram showing an example of a threshold value table in a second modification of the second embodiment;

FIG. 14 is a flowchart showing an example of a process executed by a control device in the second modification of the second embodiment;

FIG. 15 is a block diagram showing functions of a control device in a third embodiment;

FIG. 16 is a flowchart showing an example of a process executed by the control device in the third embodiment;

FIG. 17 is a diagram showing a control system in a fourth embodiment;

FIG. 18 is a sequence diagram showing an example of a process executed in the control system in the fourth embodiment;

FIG. 19 is a diagram showing a control system in a fifth embodiment; and

FIG. 20 is a sequence diagram showing an example of a process executed in the control system in the fifth embodiment.

### DETAILED DESCRIPTION OF THE INVENTION

Embodiments will be described below with reference to the drawings. The following embodiments are just examples and a variety of modifications are possible within the scope of the present disclosure.

### First Embodiment

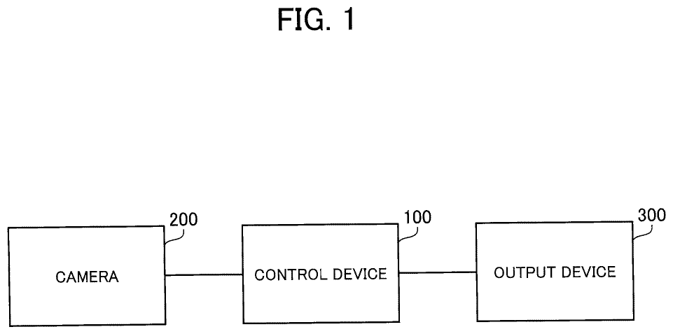

FIG. 1 is a diagram showing a control system in a first embodiment. The control system includes a control device 100, a camera 200 and an output device 300. The control device 100, the camera 200 and the output device 300 execute communication via a network.

For example, the camera 200 and the output device 300 are installed in a train station. For example, the control device 100 is installed in a place different from the camera 200 and the output device 300. A case where the camera 200 and the output device 300 are installed in a place different from the control device 100 will be shown below.

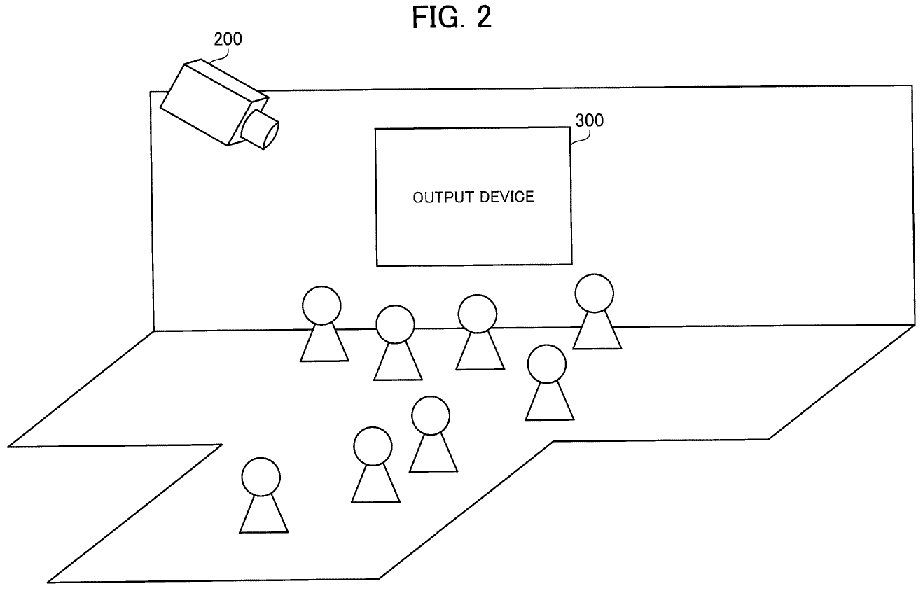

FIG. 2 is a diagram showing an installation example of the camera and the output device in the first embodiment. FIG. 2 shows the camera 200 and the output device 300. FIG. 2 shows a plurality of people existed in a place where movement in a plurality of directions is possible. Passages exist to the right and left of the output device 300 in FIG. 2. In short, there is a T-junction. The plurality of people can move to the passages. Incidentally, the place where movement in a plurality of directions is possible can also be a crossroads or the like.

The control device 100 is a device that executes a control method. The control device 100 controls the output device 300.

The camera 200 is referred to also as an image capturing device. The camera 200 can also be an infrared camera.

The output device 300 is a device that outputs a stimulus. For example, the output device 300 is a display device, a speaker, an air conditioner, a smell generator, a projector, a deodorizing machine, an illuminator, or the like. Incidentally, the display device is a digital signage, for example.

For example, the output device 300 outputs information for guiding people. In the case where the output device 300 is a projector, the output device 300 performs floor surface lighting. For example, the output device 300 is capable of guiding people by the floor surface lighting.

Here, the output device 300 in the following description is assumed to be a digital signage. Further, the following description will be given mainly of cases where a stimulus by visual sensation is changed by changing content outputted to the output device 300.

Next, hardware included in the control device 100 will be described below.

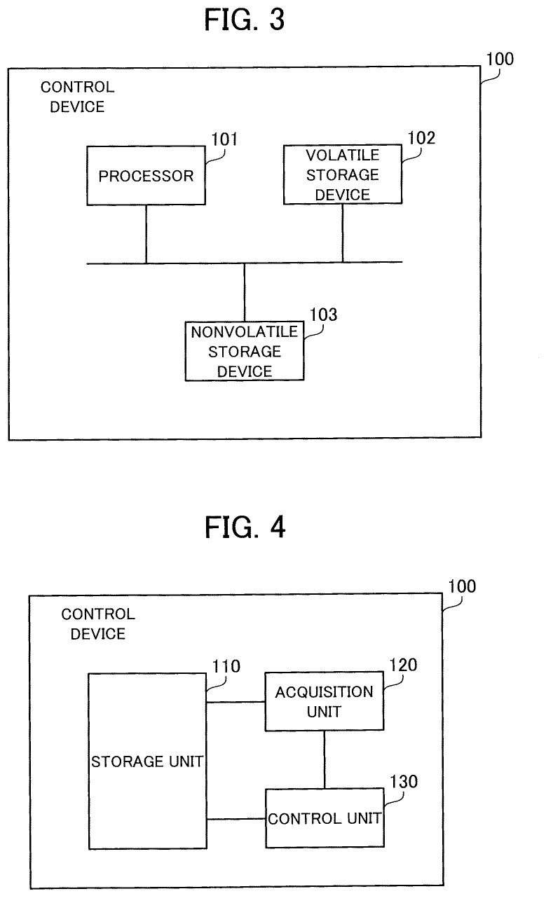

FIG. 3 is a diagram showing the hardware included in the control device in the first embodiment. The control device 100 includes a processor 101, a volatile storage device 102 and a nonvolatile storage device 103.

The processor 101 controls the whole of the control device 100. The processor 101 is a Central Processing Unit (CPU), a Field Programmable Gate Array (FPGA) or the like, for example. The processor 101 can also be a multiprocessor. Further, the control device 100 may include processing circuitry. The processing circuitry may be either a single circuit or a combined circuit.

The volatile storage device 102 is main storage of the control device 100. The volatile storage device 102 is a Random Access Memory (RAM), for example. The nonvolatile storage device 103 is auxiliary storage of the control device 100. The nonvolatile storage device 103 is a Hard Disk Drive (HDD) or a Solid State Drive (SSD), for example.

Next, functions included in the control device 100 will be described below.

FIG. 4 is a block diagram showing the functions of the control device in the first embodiment. The control device 100 includes a storage unit 110, an acquisition unit 120 and a control unit 130.

The storage unit 110 may be implemented as a storage area reserved in the volatile storage device 102 or the nonvolatile storage device 103.

Part or all of the acquisition unit 120 and the control unit 130 may be implemented by processing circuitry. Further, part or all of the acquisition unit 120 and the control unit 130 may be implemented as modules of a program executed by the processor 101. For example, the program executed by the processor 101 is referred to also as a control program. The control program has been recorded in a record medium, for example.

The functions of the acquisition unit 120 and the control unit 130 will be described in detail later.

Next, a process executed by the control device 100 will be described below by using a flowchart.

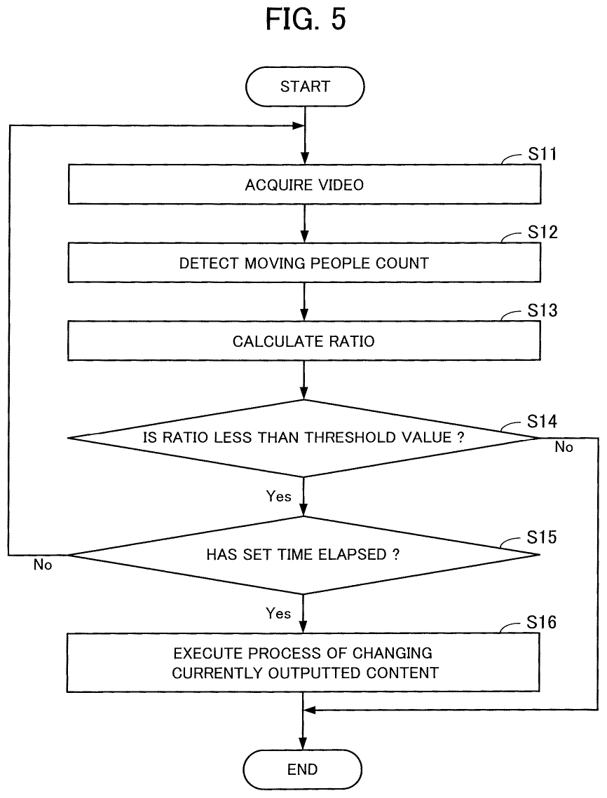

FIG. 5 is a flowchart showing an example of the process executed by the control device in the first embodiment. At the time point of starting the process of FIG. 5, the output device 300 is outputting content. Incidentally, the content can be letters, an image or a video. For example, the image can be a symbol such as an arrow.

(Step S11) The acquisition unit 120 acquires a video obtained by the camera 200 by capturing images of a plurality of people. For example, the acquisition unit 120 acquires the video from the camera 200. The acquisition unit 120 may also acquire the video from a device other than the camera 200. The video is a video obtained by the camera 200 by performing the image capturing for a predetermined time. The time is one minute, for example.

(Step S12) The control unit 130 detects a moving people count as the number of people who moved in a first direction among the plurality of directions based on the video. For example, the control unit 130 identifies the people by using the image recognition technology. The control unit 130 detects the moving people count by identifying the people. Incidentally, the first direction is, for example, a leftward direction from the output device 300 in FIG. 2.

(Step S13) The control unit 130 calculates a ratio of the people who moved in the first direction by using the moving people count. For example, the control unit 130 calculates the ratio by using the moving people count and the actual number of people included in the video. Accordingly, the ratio of the people who moved in the first direction among the actual number of people is calculated.

(Step S14) The control unit 130 judges whether or not the ratio is less than a predetermined threshold value. Here, the threshold value may be defined as follows. For example, the threshold value is “8” in “8:2”. Incidentally, “8:2” indicates that 80% of the people are guided in the leftward direction and 20% of the people are guided in the rightward direction. For example, when the threshold value is “8”, the control unit 130 judges whether or not the ratio is less than “8”.

If the ratio is less than the threshold value, the process advances to step S15. If the ratio is higher than or equal to the threshold value, the process ends.

(Step S15) The control unit 130 judges whether or not a predetermined set time has elapsed since the first judgment that the ratio is less than the threshold value. The set time is 5 minutes, for example.

If the set time has elapsed, the control unit 130 judges that the situation where the ratio is less than the threshold value is continuing for the set time. Then, the process advances to step S16. If the set time has not elapsed, the process advances to the step S11.

(Step S16) The control unit 130 executes control for changing the content currently outputted by the output device 300 to different content. For example, the control unit 130 transmits the different content and an output command for the different content to the output device 300. Accordingly, the content outputted to the output device 300 is changed.

As above, when the situation where the ratio is less than the predetermined threshold value is continuing for the set time predetermined, the control unit 130 executes the control for changing the stimulus currently outputted by the output device 300 to a different stimulus.

Here, a description will be given of a process executed by the control unit 130 in cases where the output device 300 is a speaker or the like.

In cases where the output device 300 is a speaker, the control unit 130 executes the following process. When it is judged that the situation where the ratio is less than the threshold value is continuing for the set time, the control unit 130 executes control for changing sound currently outputted by the speaker to different sound. Accordingly, the sound outputted from the speaker is changed.

In cases where the output device 300 is an air conditioner, the control unit 130 executes the following process. When it is judged that the situation where the ratio is less than the threshold value is continuing for the set time, the control unit 130 executes control for changing the direction of air currently outputted by the air conditioner to a different direction. Accordingly, the direction of the air is changed. It is also possible for the control unit 130 to execute control for changing wind power of the air conditioner.

In cases where the output device 300 is a smell generator, the control unit 130 executes the following process. When it is judged that the situation where the ratio is less than the threshold value is continuing for the set time, the control unit 130 executes control for changing a smell currently outputted by the smell generator to a different smell. Accordingly, the smell outputted from the smell generator is changed. In cases where a deodorizing machine is connected to the control device 100, the control unit 130 may activate the deodorizing machine instead of generating a different smell.

In cases where the output device 300 is a projector, the control unit 130 executes the following process. When it is judged that the situation where the ratio is less than the threshold value is continuing for the set time, the control unit 130 executes control for changing information currently outputted by the projector to different information. Accordingly, the information outputted by the projector is changed.

In cases where the output device 300 is an illuminator, the control unit 130 executes the following process. When it is judged that the situation where the ratio is less than the threshold value is continuing for the set time, the control unit 130 executes control for changing illuminance, illumination color or an illumination angle of light currently outputted by the illuminator. Accordingly, the illuminance, the illumination color or the illumination angle is changed.

Further, the control unit 130 may execute control for changing the stimulus outputted by the output device 300 to a stimulus that guides people in the first direction. For example, in cases where the output device 300 is a display device, the control unit 130 executes control for changing the content currently outputted by the display device to content that guides people in the first direction. For example, in cases where the output device 300 is a projector, the control unit 130 executes control for changing the information currently outputted by the projector to information that guides people in the first direction. For example, in cases where the output device 300 is a speaker, the control unit 130 executes control for changing the sound currently outputted by the projector to sound that guides people in the first direction. As above, the control device 100 is capable of guiding people in the first direction by the control executed by the control unit 130.

Here, as people get accustomed to a stimulus, the people stop responding to the stimulus. According to the first embodiment, the control device 100 changes the stimulus when it is judged that the situation where the ratio is less than the threshold value is continuing for the set time. For example, the control device 100 executes control for changing the content outputted to the output device 300 when it is judged that the situation where the ratio is less than the threshold value is continuing for the set time. By doing this, the stimulus by visual sensation is changed. By changing the stimulus, people begin to respond to the stimulus. Accordingly, the control device 100 is capable of making people respond to a stimulus. The above description has been given of a case where the moving people count is detected based on a video. It is also possible for the control device 100 to execute the following process. The acquisition unit 120 acquires position information regarding one or more persons from one or more terminal devices (e.g., smartphones). The control unit 130 detects the moving people count based on the position information regarding one or more persons.

### First Modification of First Embodiment

In a first modification of the first embodiment, a description will be given of a case where a stimulus dissimilar to the stimulus currently outputted is determined based on a management table. First, an example of the management table will be shown below.

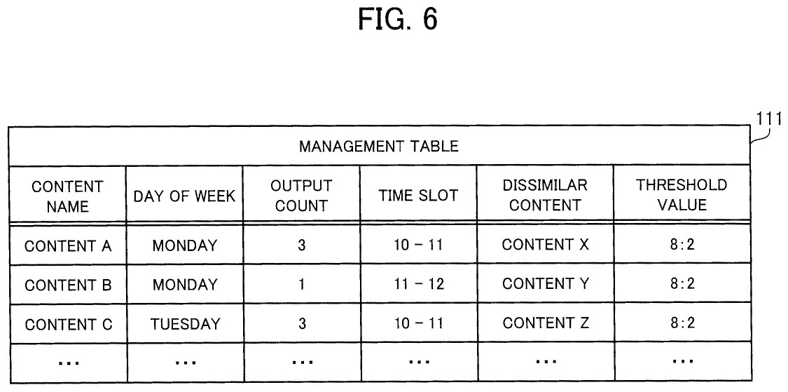

FIG. 6 is a diagram showing an example of the management table in a first modification of the first embodiment. The management table 111 may be stored in the storage unit 110. The management table 111 is referred to also as management information. The management table 111 includes items of content name, day of week, output count, time slot, dissimilar content, and threshold value.

In the item of content name, the name of content is registered. In the item of day of week, a day of the week on which the content is outputted is registered. In the item of output count, the number of times the content has been outputted is registered. In the item of time slot, a time slot in which the content is outputted is registered. In the item of dissimilar content, the name of dissimilar content is registered. For example, the content X is dissimilar to the content A. In the item of threshold value, information regarding the threshold value is registered.

As above, the management table 111 indicates a correspondence relationship between content and content dissimilar to the content. That is, the management table 111 indicates information regarding content having the same attribute. Incidentally, the similarity between content and content may be represented by a numerical value.

FIG. 6 indicates examples of content. Information regarding a stimulus other than content may also be registered in the management table 111. For example, the management table 111 may also indicate a correspondence relationship between sound and sound dissimilar to the sound.

The management table 111 is acquired by the acquisition unit 120. For example, the acquisition unit 120 acquires the management table 111 from the storage unit 110. Here, the management table 111 may be stored in an external device (e.g., cloud server). In the case where the management table 111 is stored in an external device, the acquisition unit 120 acquires the management table 111 from the external device.

Further, a threshold value has been associated with each stimulus. For example, the threshold value has been associated with content as a stimulus by visual sensation. Incidentally, the threshold value does not have to be a fixed value. The threshold value may be changed by feeding back a result obtained by outputting the content.

Next, a process executed by the control device 100 will be described below by using a flowchart.

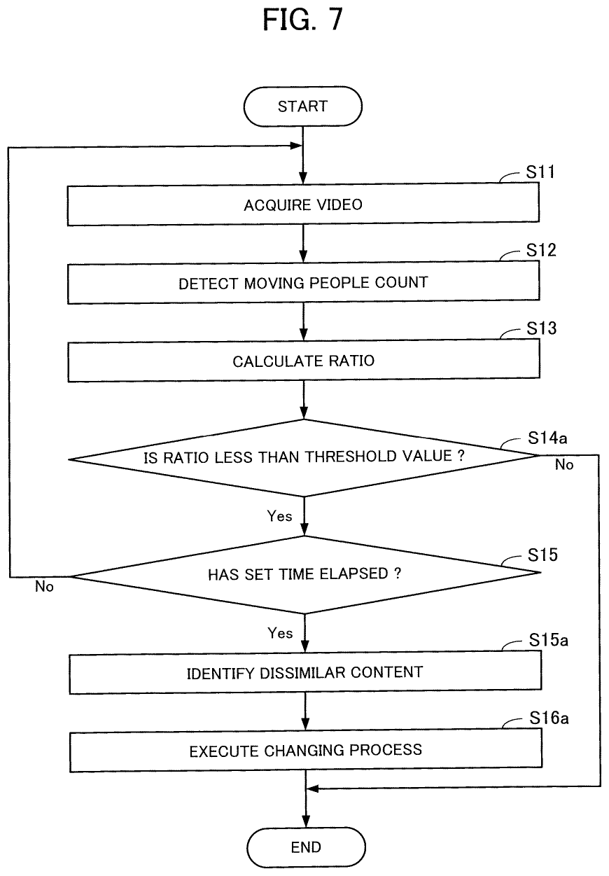

FIG. 7 is a flowchart showing an example of the process executed by the control device in the first modification of the first embodiment. The process in FIG. 7 differs from the process in FIG. 5 in that steps S14a, S15a and S16a are executed. Thus, the steps S14a, S15a and S16a in FIG. 7 will be described below. Then, the description will be omitted for processing other than the steps S14a, S15a and S16a.

(Step S14a) The control unit 130 judges whether or not the ratio is less than the threshold value corresponding to the content currently outputted.

If the ratio is less than the threshold value, the process advances to the step S15. If the ratio is higher than or equal to the threshold value, the process ends.

(Step S15a) The control unit 130 identifies the dissimilar stimulus based on the currently outputted stimulus and the management table 111. Specifically, the control unit 130 identifies content dissimilar to the currently outputted content based on the currently outputted content and the management table 111. For example, when the currently outputted content is the content A, the control unit 130 identifies the content X. It is also possible for the control unit 130 to execute the following process. The control unit 130 identifies content dissimilar to the currently outputted content based on the threshold value corresponding to the currently outputted content and the management table 111. For example, the control unit 130 refers to the management table 111 and identifies records of the threshold value corresponding to the currently outputted content. The control unit 130 identifies a record of the currently outputted content A out of the identified records. The control unit 130 identifies the content X in the record of the content A.

(Step S16a) The control unit 130 executes control for changing the currently outputted stimulus to a dissimilar stimulus. Specifically, the control unit 130 executes control for changing the currently outputted content to the identified dissimilar content. For example, the control unit 130 transmits the dissimilar content and an output command for the dissimilar content to the output device 300. Accordingly, the content outputted to the output device 300 is changed.

By changing the content in the process of FIG. 7 to sound, wind, smell, information outputted by the projector, or lighting, dissimilar sound, wind, smell, information outputted by the projector, or lighting is outputted. In short, a dissimilar stimulus is outputted.

According to the first modification of the first embodiment, the control device 100 is capable of making people more responsive to a stimulus by changing the stimulus to a dissimilar stimulus.

### Second Modification of First Embodiment

In a second modification of the first embodiment, a description will be given of a case where the threshold value is changed based on a user attribute. When the threshold value is changed, a threshold value table is used. An example of the threshold value table will be shown below.

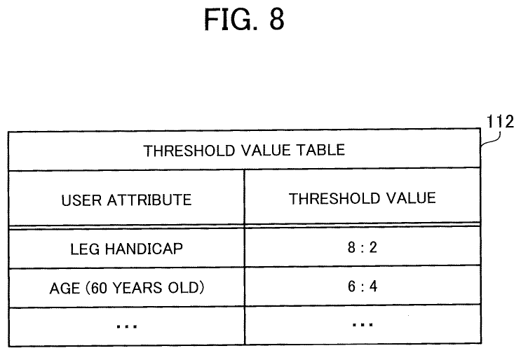

FIG. 8 is a diagram showing an example of the threshold value table in the second modification of the first embodiment. The threshold value table 112 may be stored in the storage unit 110. The threshold value table 112 is referred to also as threshold value information. The threshold value table 112 includes items of user attribute and threshold value.

In the item of user attribute, information indicating the user attribute is registered. In the item of threshold value, information regarding the threshold value is registered.

The threshold value table 112 is acquired by the acquisition unit 120. For example, the acquisition unit 120 acquires the threshold value table 112 from the storage unit 110. Here, the threshold value table 112 may be stored in an external device. In the case where the threshold value table 112 is stored in an external device, the acquisition unit 120 acquires the threshold value table 112 from the external device.

Further, content and the threshold value table 112 may be associated with each other. In the case where content and the threshold value table 112 are associated with each other, the acquisition unit 120 acquires a threshold value table 112 corresponding to the currently outputted content. As above, each of a plurality of pieces of content may be associated with a threshold value table 112.

Next, a process executed by the control device 100 will be described below by using a flowchart.

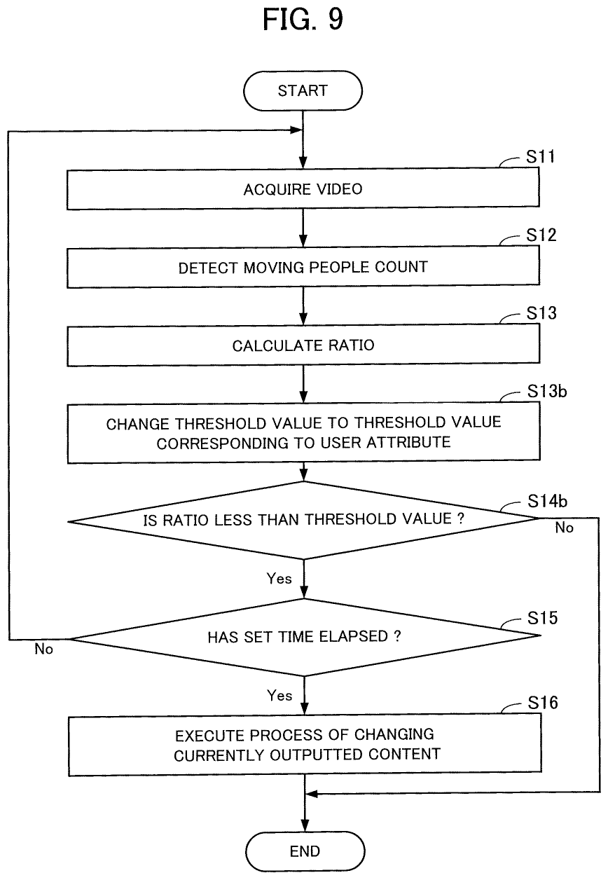

FIG. 9 is a flowchart showing an example of the process executed by the control device in the second modification of the first embodiment. The process in FIG. 9 differs from the process in FIG. 5 in that steps S13b and S14b are executed. Thus, the steps S13b and S14b in FIG. 9 will be described below. Then, the description will be omitted for processing other than the steps S13b and S14b.

(Step S13b) The control unit 130 identifies a user attribute as an attribute of a person included in the video based on the video. Specifically, the control unit 130 identifies the user attribute by using the video and the image recognition technology. For example, the control unit 130 detects a wheelchair based on the video and thereby identifies that there exists a person having a leg handicap in the video. Further, for example, based on the video, the control unit 130 identifies that the age of a person existing in the video is “60 years old”.

The control unit 130 identifies the threshold value corresponding to the user attribute based on the threshold value table 112. For example, the control unit 130 identifies the threshold value “6” corresponding to “60 years old” based on the threshold value table 112.

The control unit 130 changes the predetermined threshold value to the threshold value corresponding to the user attribute. The control unit 130 may identify a user attribute having the highest ratio among a plurality of user attributes based on the plurality of people included in the video. Then, the control unit 130 may identify the threshold value corresponding to the identified user attribute based on the threshold value table 112.

(Step S14b) The control unit 130 judges whether or not the ratio is less than the threshold value corresponding to the user attribute.

If the ratio is less than the threshold value, the process advances to the step S15. If the ratio is higher than or equal to the threshold value, the process ends.

According to the second modification of the first embodiment, the control device 100 is capable of making a judgment dependent on the attribute of a person included in the video by making the judgment by using the threshold value corresponding to the user attribute.

### Second Embodiment

Next, a second embodiment will be described below. In the second embodiment, the description will be given mainly of features different from those in the first embodiment. In the second embodiment, the description is omitted for features in common with the first embodiment. FIGS. 1 to 3 and 6 are referred to in the second embodiment.

In the first embodiment, the description has given of the case where the content is changed based on the ratio of the people who moved in the first direction. In the second embodiment, a description will be given of a case where the content is changed based on a visual recognition rate.

FIG. 10 is a block diagram showing functions of a control device in the second embodiment. The control device 100a controls the output device 300 that outputs information. In the second embodiment, the output device 300 is a display device or a projector.

The control device 100a includes a storage unit 110a, an acquisition unit 120a and a control unit 130a.

The storage unit 110a may be implemented as a storage area reserved in the volatile storage device 102 or the nonvolatile storage device 103.

Part or all of the acquisition unit 120a and the control unit 130a may be implemented by processing circuitry. Further, part or all of the acquisition unit 120a and the control unit 130a may be implemented as modules of a program executed by the processor 101. For example, the program executed by the processor 101 is referred to also as a control program. The control program has been recorded in a record medium, for example.

The acquisition unit 120a acquires an image obtained by the camera 200 by capturing an image of a plurality of people. For example, the acquisition unit 120a acquires the image from the camera 200. The acquisition unit 120a may also acquire the image from a device other than the camera 200.

The control unit 130a calculates the visual recognition rate regarding the information currently outputted by the output device 300 based on the image. For example, in the case where the output device 300 is a display device, the control unit 130a calculates the visual recognition rate regarding the content currently outputted by the display device. Further, for example, in the case where the output device 300 is a projector, the control unit 130a calculates the visual recognition rate regarding the information currently outputted by the projector. When the visual recognition rate is lower than a predetermined threshold value, the control unit 130a executes control for changing the information currently outputted by the output device 300 to different information. For example, in the case where the output device 300 is a display device, the control unit 130a executes control for changing the content currently outputted by the display device to different content. Further, for example, in the case where the output device 300 is a projector, the control unit 130a executes control for changing the information currently outputted by the projector to different information.

Next, a process executed by the control device 100a will be described below by using a flowchart.

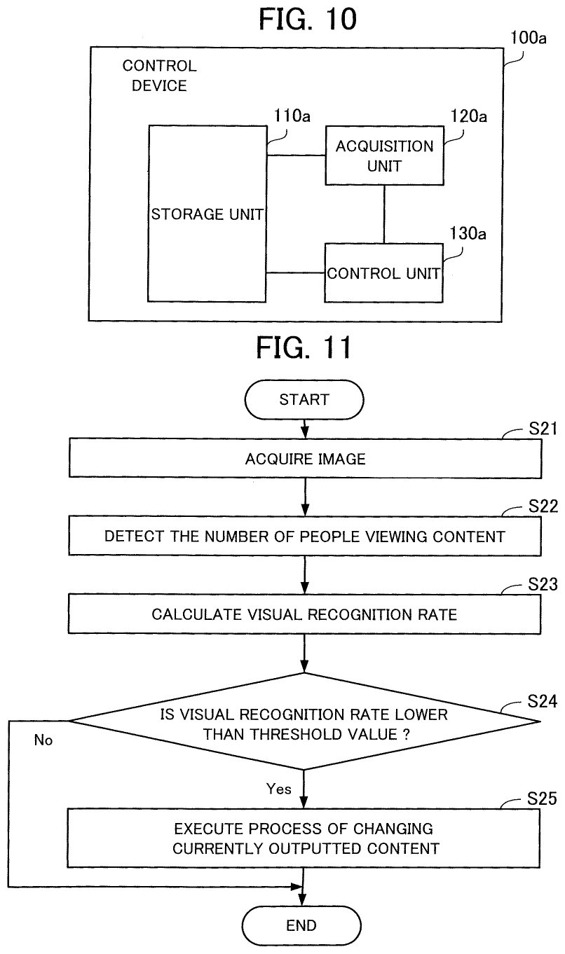

FIG. 11 is a flowchart showing an example of the process executed by the control device in the second embodiment. At the time point of starting the process of FIG. 11, the output device 300 is outputting content.

(Step S21) The acquisition unit 120a acquires the image from the camera 200.

(Step S22) The control unit 130a detects a people count as the number of people viewing the content based on the image. For example, the control unit 130a detects the people count based on the direction of the face of each person included in the image or the line of sight of each person included in the image.

It is also possible for the control unit 130a to detect the people count by subtracting the number of people not viewing the content from the total number of people in the image.

(Step S23) The control unit 130a calculates the visual recognition rate based on the detected people count and the total number of people in the image.

(Step S24) The control unit 130a judges whether or not the calculated visual recognition rate is lower than the predetermined threshold value. Incidentally, the threshold value can also be a visual recognition rate measured in the past, for example.

If the visual recognition rate is lower than the threshold value, the process advances to step S25. If the visual recognition rate is higher than or equal to the threshold value, the process ends.

(Step S25) The control unit 130a executes control for changing the currently outputted content to different content. For example, the control unit 130a transmits the different content and an output command for the different content to the output device 300. Accordingly, the content outputted to the output device 300 is changed.

Further, the acquisition unit 120a may acquire the management table 111. The management table 111 indicates a correspondence relationship between the currently outputted information and the dissimilar information. Then, the control unit 130a may identify the dissimilar information based on the currently outputted information and the management table 111 and execute the control for changing the currently outputted information to the dissimilar information. For example, the control unit 130a identifies the dissimilar content based on the currently outputted content and the management table 111. The control unit 130a executes the control for changing the currently outputted content to the dissimilar content.

Here, for example, when the visual recognition rate is lower than the threshold value, it can be considered that people are not viewing the content since the people have got accustomed to the content. That is, it can be considered that the people have got accustomed to the stimulus by visual sensation. Therefore, when the visual recognition rate is lower than the threshold value, the control device 100a executes the control for changing the currently outputted content to different content. In other words, the control device 100a executes control for changing the stimulus by visual sensation. By doing this, people begin to view the content. Accordingly, the control device 100a is capable of making people respond to a stimulus.

### First Modification of Second Embodiment

In the second embodiment, the description has given of the case where an image is acquired. In a first modification of the second embodiment, a description will be given of a case where a video is acquired.

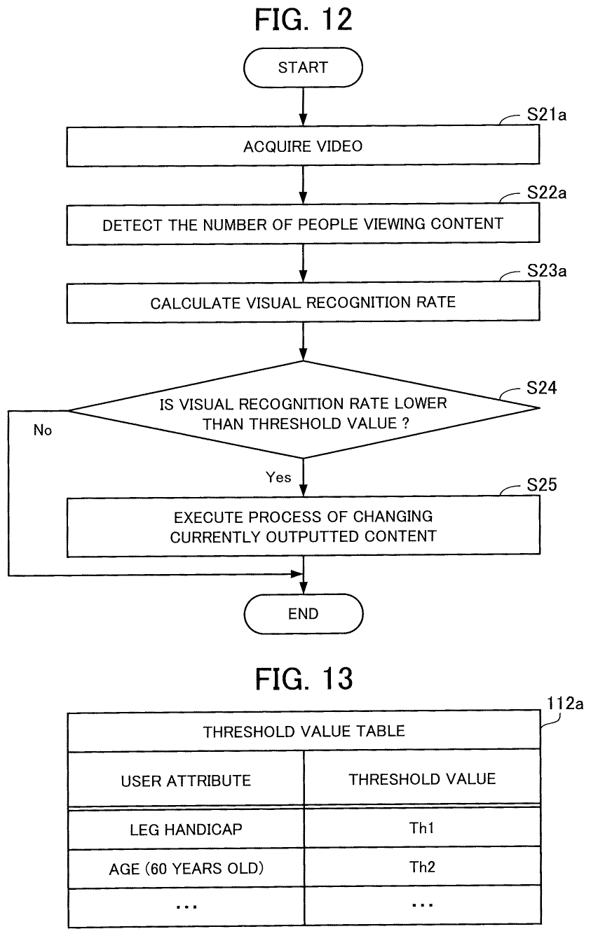

FIG. 12 is a flowchart showing an example of a process executed by a control device in the first modification of the second embodiment. The process in FIG. 12 differs from the process in FIG. 11 in that steps S21a, S22a and S23a are executed. Thus, the steps S21a, S22a and S23a in FIG. 12 will be described below. Then, the description will be omitted for processing other than the steps S21a, S22a and S23a.

(Step S21a) The acquisition unit 120a acquires a video from the camera 200. For example, the acquisition unit 120a acquires the video from the camera 200. The acquisition unit 120a may also acquire the video from a device other than the camera 200. The video is a video obtained by the camera 200 by performing the image capturing for a predetermined time. The time is one minute, for example.

(Step S22a) The control unit 130a detects a people count as the number of people viewing the content for a predetermined time based on the video. For example, the control unit 130a detects the people count as the number of people viewing the content for three seconds among the people included in the video.

(Step S23a) The control unit 130a calculates the visual recognition rate based on the detected people count and the actual number of people included in the video.

It is also possible for the control unit 130a to calculate an average value of a plurality of visual recognition rates, respectively obtained by calculation based on each of a plurality of images as the video, as the visual recognition rate.

According to the first modification of the second embodiment, the control device 100a is capable of making people respond to a stimulus.

### Second Modification of Second Embodiment

In a second modification of the second embodiment, a description will be given of a case where the threshold value is changed based on the user attribute. When the threshold value is changed, a threshold value table is used. An example of the threshold value table will be shown below.

FIG. 13 is a diagram showing an example of the threshold value table in the second modification of the second embodiment. The threshold value table 112a may be stored in the storage unit 110a. The threshold value table 112a is referred to also as threshold value information. The threshold value table 112a includes items of user attribute and threshold value.

In the item of user attribute, information indicating the user attribute is registered. In the item of threshold value, information regarding the threshold value is registered.

The threshold value table 112a is acquired by the acquisition unit 120a. For example, the acquisition unit 120a acquires the threshold value table 112a from the storage unit 110a. Here, the threshold value table 112a may be stored in an external device. In the case where the threshold value table 112a is stored in an external device, the acquisition unit 120a acquires the threshold value table 112a from the external device. Next, a process executed by the control device 100a will be described below by using a flowchart.

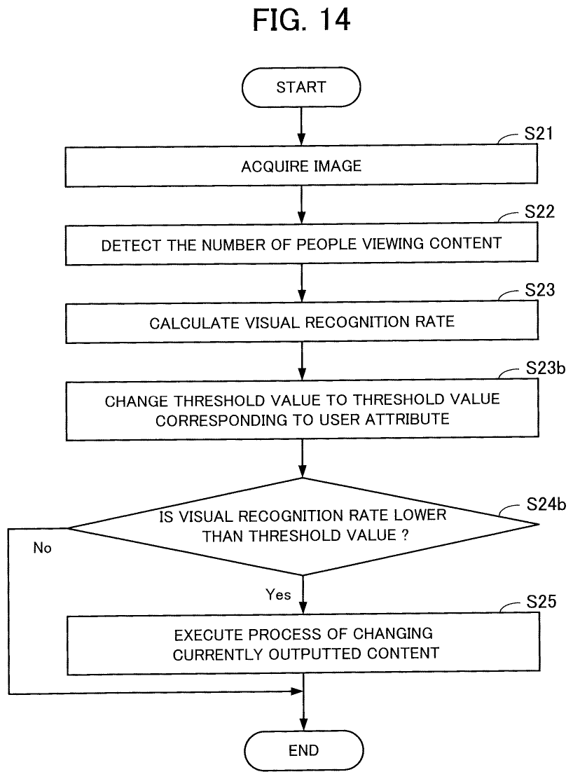

FIG. 14 is a flowchart showing an example of the process executed by the control device in the second modification of the second embodiment. The process in FIG. 14 differs from the process in FIG. 11 in that steps S23b and S24b are executed. Thus, the steps S23b and S24b in FIG. 14 will be described below. Then, the description will be omitted for processing other than the steps S23b and S24b.

(Step S23b) The control unit 130a identifies a user attribute as an attribute of a person included in the image based on the image. Specifically, the control unit 130a identifies the user attribute by using the image and the image recognition technology. For example, based on the image, the control unit 130a identifies that the age of a person existing in the image is “60 years old”.

The control unit 130a identifies the threshold value corresponding to the user attribute based on the threshold value table 112a. For example, the control unit 130a identifies the threshold value “Th2” corresponding to “60 years old” based on the threshold value table 112a.

The control unit 130a changes the predetermined threshold value to the threshold value corresponding to the user attribute.

(Step S24b) The control unit 130a judges whether or not the calculated visual recognition rate is lower than the threshold value corresponding to the user attribute.

If the visual recognition rate is lower than the threshold value, the process advances to step S25. If the visual recognition rate is higher than or equal to the threshold value, the process ends.

While the above description has given of the case where the threshold value is changed when an image is acquired, the threshold value may also be changed when a video is acquired.

According to the second modification of the second embodiment, the control device 100a is capable of making a judgment dependent on the attribute of a person included in the image or video by making the judgment by using the threshold value corresponding to the user attribute.

### Third Embodiment

Next, a third embodiment will be described below. In the third embodiment, the description will be given mainly of features different from those in the first or second embodiment. In the third embodiment, the description is omitted for features in common with the first or second embodiment. In the third embodiment, the output device 300 is a display device or a projector.

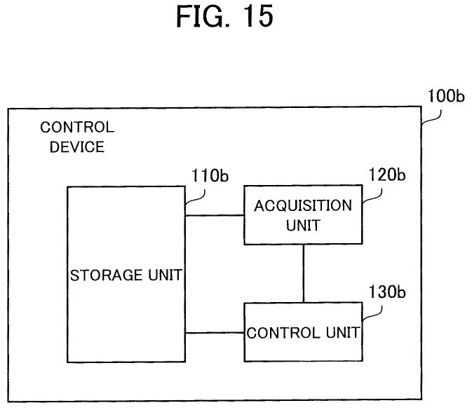

FIG. 15 is a block diagram showing functions of a control device in the third embodiment. The control device 100b includes a storage unit 110b, an acquisition unit 120b and a control unit 130b.

The storage unit 110b may be implemented as a storage area reserved in the volatile storage device 102 or the nonvolatile storage device 103.

Part or all of the acquisition unit 120b and the control unit 130b may be implemented by processing circuitry. Further, part or all of the acquisition unit 120b and the control unit 130b may be implemented as modules of a program executed by the processor 101. For example, the program executed by the processor 101 is referred to also as a control program. The control program has been recorded in a record medium, for example.

The functions of the acquisition unit 120b and the control unit 130b will be described in detail later.

Next, a process executed by the control device 100b will be described below by using a flowchart.

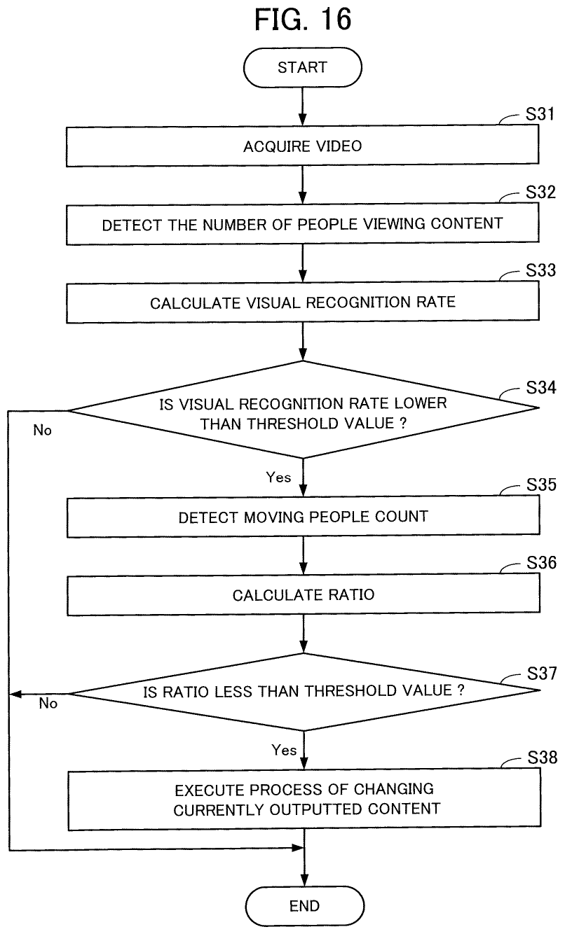

FIG. 16 is a flowchart showing an example of the process executed by the control device in the third embodiment. At the time point of starting the process of FIG. 16, the output device 300 is outputting content. Further, as shown in FIG. 2, a plurality of people are existed in a place where movement in a plurality of directions is possible.

(Step S31) The acquisition unit 120b acquires a video obtained by the camera 200 by capturing images of a plurality of people.

(Step S32) The control unit 130b detects the people count as the number of people viewing the content for a predetermined time based on the video. For example, the control unit 130b detects the people count as the number of people viewing the content for three seconds among the people included in the video.

(Step S33) The control unit 130b calculates the visual recognition rate based on the detected people count and the actual number of people included in the video.

It is also possible for the control unit 130b to calculate the average value of a plurality of visual recognition rates, respectively obtained by calculation based on each of a plurality of images as the video, as the visual recognition rate.

(Step S34) The control unit 130b judges whether or not the calculated visual recognition rate is lower than the predetermined threshold value.

If the visual recognition rate is lower than the threshold value, the process advances to step S35. If the visual recognition rate is higher than or equal to the threshold value, the process ends.

(Step S35) The control unit 130b detects the moving people count as the number of people who moved in the first direction among the plurality of directions based on the video. Here, the first direction is the leftward direction, for example.

(Step S36) The control unit 130b calculates the ratio of the people who moved in the first direction by using the moving people count.

(Step S37) The control unit 130b judges whether or not the ratio is less than the predetermined threshold value.

If the ratio is less than the threshold value, the process advances to step S38. If the ratio is higher than or equal to the threshold value, the process ends.

(Step S38) The control unit 130b executes the control for changing the information currently outputted by the output device 300 to different information. Specifically, the control unit 130b executes control for changing the currently outputted content to different content.

It is also possible for the control unit 130b to execute the control for changing the information currently outputted by the output device 300 to different information when the visual recognition rate is higher than or equal to the threshold value and the ratio is less than the threshold value.

According to the third embodiment, the control device 100b judges whether the content should be changed or not by using the visual recognition rate and the ratio based on the moving people count. Therefore, the control device 100b is capable of giving a stimulus by visual sensation depending on detailed conditions of the people existing in the video.

Further, in the second and third embodiments, the description has given of the cases where the control for changing the information (e.g., content) currently outputted by the output device 300 to different information is executed. The control regarding the sound, wind, smell or illumination may also be executed similarly to the first modification of the first embodiment. The control will be described in detail below. The control device 100b communicates with a display device that displays information (e.g., content). Further, the control device 100b controls an output device that outputs a stimulus (e.g., sound, wind, smell or illumination). Incidentally, the output device is installed in the vicinity of the display device. Further, the output device may be arranged at a fixed position in the vicinity of the display device. The control unit 130b calculates a visual recognition rate, as a ratio at which the information displayed by the display device is visually recognized thanks to the stimulus outputted by the output device, based on the image or video acquired by the acquisition unit 120b. When the visual recognition rate is lower than a predetermined threshold value, the control unit 130b executes control for changing the stimulus currently outputted by the output device to a different stimulus. For example, the control unit 130b executes control for changing sound currently outputted by the output device to sound that prompts the people to view the display device more. As above, the visual recognition rate is increased by changing the stimulus outputted by the output device.

Furthermore, the management table 111 acquired by the acquisition unit 120b may indicate a correspondence relationship between the currently outputted stimulus and a dissimilar stimulus. Then, the dissimilar stimulus may be identified based on the management table 111.

In the first to third embodiments, the output device 300 may be included in the control device 100, 100a, 100b. For example, the control device 100, 100a, 100b includes a display device or a projector. In cases where the output device 300 is a display device, the control unit 130 executes control for outputting content different from the content currently outputted by the display device to the display device.

### Fourth Embodiment

Next, a fourth embodiment will be described below. In the fourth embodiment, the description will be given mainly of features different from those in the second embodiment. In the fourth embodiment, the description is omitted for features in common with the second embodiment.

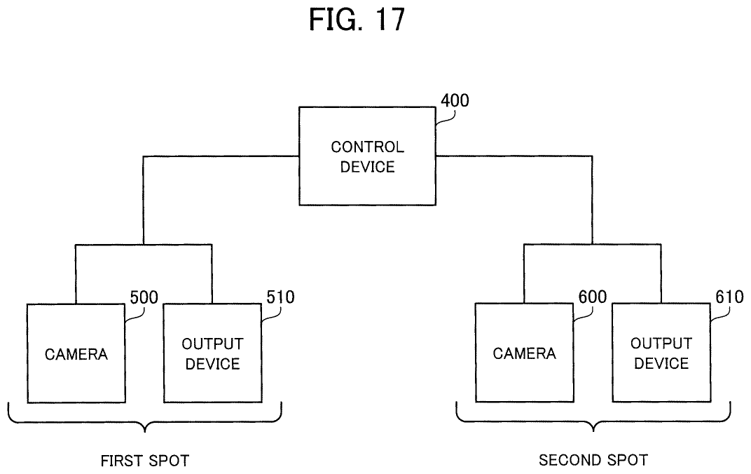

FIG. 17 is a diagram showing a control system in the fourth embodiment. The control system includes a control device 400, a camera 500, an output device 510, a camera 600 and an output device 610.

The camera 500 is referred to also as a first image capturing device. The output device 510 is referred to also as a first output device. The camera 600 is referred to also as a second image capturing device. The output device 610 is referred to also as a second output device. Each of the output device 510 and the output device 610 is a display device or a projector.

The control device 400, the camera 500, the output device 510, the camera 600 and the output device 610 are connected together via a network.

The camera 500 and the output device 510 are installed at a first spot. The camera 600 and the output device 610 are installed at a second spot. The second spot is a spot after the first spot. For example, when a person passes through the second spot subsequently to the first spot, the person views the content outputted by the output device 610 after viewing the content outputted by the output device 510.

The control device 400 manages the content outputted by the output device 510 and the content outputted by the output device 610.

Next, a process executed in the control system will be described below by using a sequence diagram.

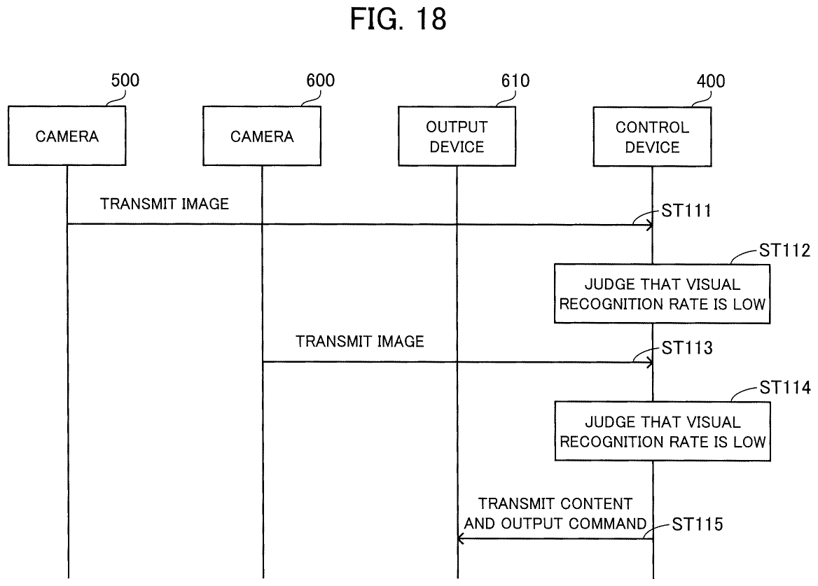

FIG. 18 is a sequence diagram showing an example of the process executed in the control system in the fourth embodiment.

(Step ST111) The camera 500 transmits an image obtained by capturing an image of a plurality of people to the control device 400.

(Step ST112) The control device 400 calculates the visual recognition rate by using the image. The calculation method of the visual recognition rate is the same as the calculation method of the visual recognition rate in the second embodiment. The control device 400 judges that the visual recognition rate is lower than a predetermined threshold value. Incidentally, this visual recognition rate is referred to also as a first visual recognition rate.

(Step ST113) The camera 600 transmits an image obtained by capturing an image of a plurality of people to the control device 400.

(Step ST114) The control device 400 calculates the visual recognition rate by using the image. The calculation method of the visual recognition rate is the same as the calculation method of the visual recognition rate in the second embodiment. The control device 400 judges that the visual recognition rate is lower than a predetermined threshold value. Incidentally, this visual recognition rate is referred to also as a second visual recognition rate.

(Step ST115) If the visual recognition rate is low at the first spot and the visual recognition rate is low at the second spot, the control device 400 executes control for making the output device 610 output content different from content currently outputted by the output device 510. Specifically, the control device 400 transmits the different content and an output command for the different content to the output device 610.

Accordingly, the different content is outputted to the output device 610.

The above description has given of the case where the visual recognition rates are calculated by using images. It is also possible for the control device 400 to calculate the visual recognition rates by using videos transmitted by the camera 500 and the camera 600. Incidentally, the calculation method of the visual recognition rate is the same as the calculation method of the visual recognition rate in the first modification of the second embodiment.

According to the fourth embodiment, the control system is capable of making people respond to a stimulus.

It is also possible for the control device 400 to execute control for making the output device 610 output information different from information currently outputted by the output device 510 when the first visual recognition rate is lower than the predetermined threshold value. By this control, even when a person did not view the content at the first spot, the person becomes likely to view the content at the second spot. Thus, according to the fourth embodiment, the control system is capable of making people respond to a stimulus.

### Fifth Embodiment

Next, a fifth embodiment will be described below. In the fifth embodiment, the description will be given mainly of features different from those in the second or fourth embodiment. In the fifth embodiment, the description is omitted for features in common with the second or fourth embodiment.

In the fourth embodiment, the description has given of the case where one control device is included in the control system. In the fifth embodiment, a description will be given of a case where two control devices are included in a control system.

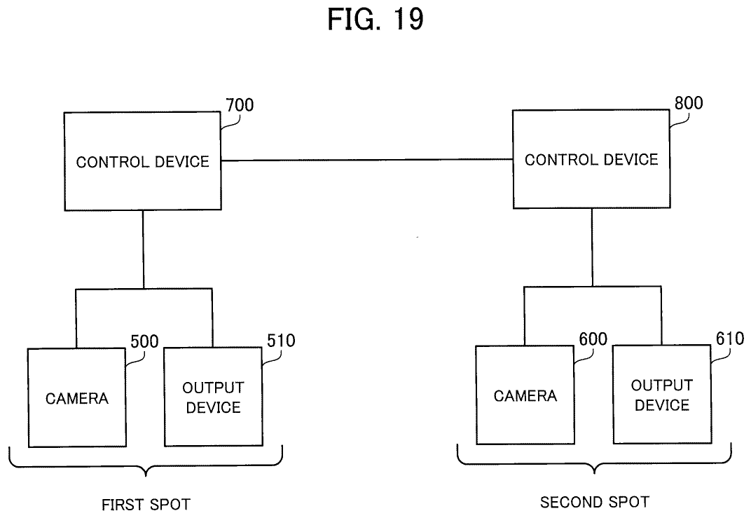

FIG. 19 is a diagram showing the control system in the fifth embodiment. The control system includes a control device 700, a control device 800, the camera 500, the output device 510, the camera 600 and the output device 610.

The control device 700 and the control device 800 are connected to each other via a network. The control device 700 is referred to also as a first control device. The control device 800 is referred to also as a second control device.

The control device 700, the camera 500 and the output device 510 are connected together by a network. The control device 700 is a device that controls the camera 500 and the output device 510. The control device 700 manages information to be outputted by the output device 510.

The control device 800, the camera 600 and the output device 610 are connected together by a network. The control device 800 is a device that controls the camera 600 and the output device 610. The control device 800 manages information to be outputted by the output device 610.

Next, a process executed in the control system will be described below by using a sequence diagram.

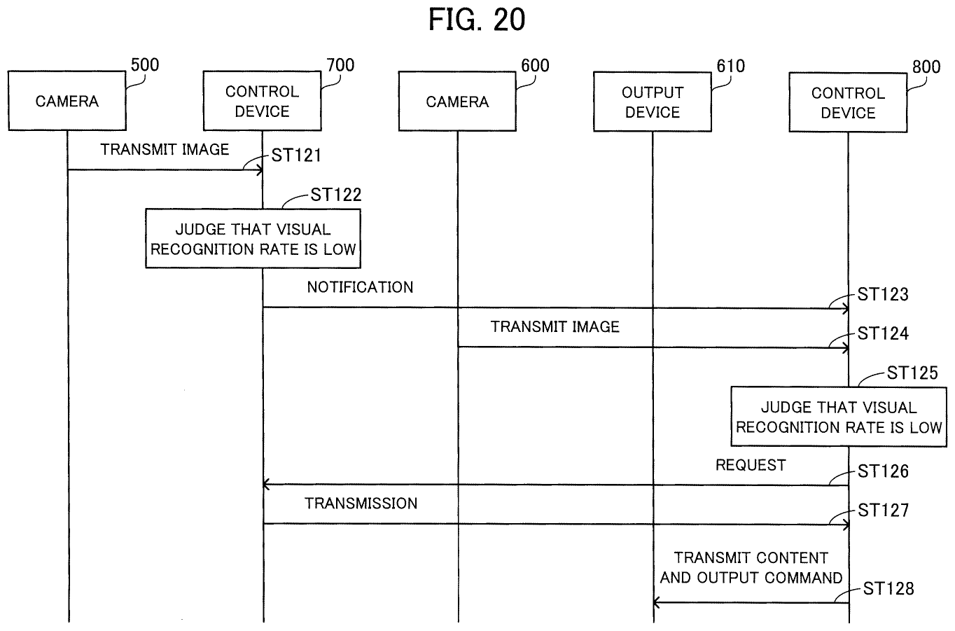

FIG. 20 is a sequence diagram showing an example of the process executed in the control system in the fifth embodiment.

(Step ST121) The camera 500 transmits an image obtained by capturing an image of a plurality of people to the control device 700.

(Step ST122) The control device 700 calculates the visual recognition rate by using the image. The calculation method of the visual recognition rate is the same as the calculation method of the visual recognition rate in the second embodiment. The control device 700 judges that the visual recognition rate is lower than a predetermined threshold value. Incidentally, this visual recognition rate is referred to also as a first visual recognition rate.

(Step ST123) The control device 700 notifies the control device 800 that the visual recognition rate is low at the first spot.

(Step ST124) The camera 600 transmits an image obtained by capturing an image of a plurality of people to the control device 800.

(Step ST125) The control device 800 calculates the visual recognition rate by using the image. The calculation method of the visual recognition rate is the same as the calculation method of the visual recognition rate in the second embodiment. The control device 800 judges that the visual recognition rate is low. Incidentally, this visual recognition rate is referred to also as a second visual recognition rate.

(Step ST126) The control device 800 transmits a transmission request, requesting transmission of information regarding the content currently outputted by the output device 510, to the control device 700.

(Step ST127) The control device 700 transmits the information regarding the content currently outputted by the output device 510 to the control device 800.

(Step ST128) The control device 800 executes control for making the output device 610 output content different from the content currently outputted by the output device 510.

Specifically, the control device 800 transmits the different content and an output command for the different content to the output device 610.

Accordingly, the different content is outputted to the output device 610.

The above description has given of the case where the visual recognition rate is calculated by using an image. It is also possible for the control device 700 to calculate the visual recognition rate by using a video transmitted by the camera 500. It is also possible for the control device 800 to calculate the visual recognition rate by using a video transmitted by the camera 600. Incidentally, the calculation method of the visual recognition rate is the same as the calculation method of the visual recognition rate in the first modification of the second embodiment.

According to the fifth embodiment, the control system is capable of making people respond to a stimulus similarly to the fourth embodiment.

It is also possible for the control device 800 to transmit a transmission request, requesting transmission of information currently outputted by the output device 510, to the control device 700, when the first visual recognition rate is lower than the threshold value. By this, even when a person did not view the content at the first spot, the person becomes likely to view the content at the second spot. Thus, according to the fifth embodiment, the control system is capable of making people respond to a stimulus.

The control device in any one of the first to fifth embodiments may also include at least one of a camera and an output device (e.g., display). Further, the functions of the control device in any one of the first to fifth embodiments may be implemented in a form of being distributed to a plurality of devices such as an input device like a camera, an output device like a display, and so forth. Furthermore, the control device may include a plurality of devices such as a camera and an output device as mentioned above.

Features in the embodiments described above can be appropriately combined with each other.

### DESCRIPTION OF REFERENCE CHARACTERS

100: control device, 100a: control device, 100b: control device, 101: processor, 102: volatile storage device, 103: nonvolatile storage device, 110: storage unit, 110a: storage unit, 110b: storage unit, 111: management table, 112: threshold value table, 112a: threshold value table, 120: acquisition unit, 120a: acquisition unit, 120b: acquisition unit, 130: control unit, 130a: control unit, 130b: control unit, 200: camera, 300: output device, 400: control device, 500: camera, 510: output device, 600: camera, 610: output device, 700: control device, 800: control device

What is claimed is:

1. A control device for controlling an output device that outputs a stimulus, the control device comprising:
acquiring circuitry to acquire a video obtained by an image capturing device by capturing images of a plurality of people existed in a place where movement in a plurality of directions is possible; and
controlling circuitry to detect a moving people count as a number of people who moved in a first direction among the plurality of directions based on the video, calculate a ratio of the people who moved in the first direction by using the moving people count, and when a situation where the ratio is less than a predetermined threshold value is continuing for a predetermined set time, execute control for changing the stimulus currently outputted by the output device to a different stimulus.

2. The control device according to claim 1, wherein
the acquiring circuitry acquires management information that indicates a correspondence relationship between the currently outputted stimulus and a dissimilar stimulus having a same attribute as the currently outputted stimulus and dissimilar to the currently outputted stimulus, and
the controlling circuitry identifies the dissimilar stimulus based on the currently outputted stimulus and the management information and executes control for changing the currently outputted stimulus to the dissimilar stimulus.

3. The control device according to claim 1, wherein
the acquiring circuitry acquires threshold value information,
the controlling circuitry identifies a user attribute as an attribute of a person included in the video based on the video and identifies a threshold value corresponding to the user attribute based on the threshold value information, and
the predetermined threshold value is the identified threshold value.

4. The control device according to claim 1, wherein when the output device is a display device, the controlling circuitry executes control for changing content currently outputted by the display device to different content.

5. The control device according to claim 1, wherein when the output device is a speaker, the controlling circuitry executes control for changing sound currently outputted by the speaker to different sound.

6. The control device according to claim 1, wherein when the output device is an air conditioner, the controlling circuitry executes at least one of control for changing a direction of air currently outputted by the air conditioner to a different direction and control for changing wind power of the air conditioner.

7. The control device according to claim 1, wherein when the output device is a smell generator, the controlling circuitry executes control for changing a smell currently outputted by the smell generator to a different smell.

8. The control device according to claim 1, wherein when the output device is a projector, the controlling circuitry executes control for changing information currently outputted by the projector to different information.

9. The control device according to claim 1, wherein when the output device is an illuminator, the controlling circuitry executes control for changing illuminance, illumination color or an illumination angle of light currently outputted by the illuminator.

10. The control device according to claim 1, wherein when the output device is a display device or a projector, the control device further comprises the display device or the projector.

11. A control device for controlling an output device that outputs a stimulus, the control device comprising:
acquiring circuitry to acquire an image or a video obtained by an image capturing device by capturing one or more images of a plurality of people and acquire management information that indicates a correspondence relationship between a currently outputted stimulus and a dissimilar stimulus; and
controlling circuitry to calculate a visual recognition rate regarding the information currently outputted by the output device based on the image or the video, when the visual recognition rate is lower than a predetermined threshold value, identify the dissimilar stimulus based on the currently outputted stimulus and the management information and execute control for changing the currently outputted stimulus to the dissimilar stimulus.

12. The control device according to claim 11, wherein
the acquiring circuitry acquires threshold value information,
the controlling circuitry identifies a user attribute as an attribute of a person included in the image or the video based on the image or the video and identifies a threshold value corresponding to the user attribute based on the threshold value information, and
the predetermined threshold value is the identified threshold value.

13. The control device according to claim 11, wherein
the plurality of people are existed in a place where movement in a plurality of directions is possible, and
when the visual recognition rate based on the video is lower than the threshold value, the controlling circuitry detects a moving people count as a number of people who moved in a first direction among the plurality of directions based on the video, calculates a ratio of the people who moved in the first direction by using the moving people count, and when the ratio is less than a predetermined threshold value, executes control for changing the currently outputted stimulus to the dissimilar stimulus.

14. The control device according to claim 11, wherein
the plurality of people are existed in a place where movement in a plurality of directions is possible, and
when the visual recognition rate based on the video is higher than or equal to the threshold value, the controlling circuitry detects a moving people count as a number of people who moved in a first direction among the plurality of directions based on the video, calculates a ratio of the people who moved in the first direction by using the moving people count, and when the ratio is less than a predetermined threshold value, executes control for changing the currently outputted stimulus to the dissimilar stimulus.

15. A control device for communicating with a display device that displays information and controlling an output device that outputs a stimulus, the control device comprising:
acquiring circuitry to acquire an image or a video obtained by an image capturing device by capturing one or more images of a plurality of people; and
controlling circuitry to calculate a visual recognition rate, as a ratio at which the information displayed by the display device is visually recognized due to the stimulus outputted by the output device, based on the image or the video, and when the visual recognition rate is lower than a predetermined threshold value, execute control for changing the stimulus currently outputted by the output device to a different stimulus without changing the information displayed by the display device.

16. The control device according to claim 15, wherein when the output device is a speaker, the controlling circuitry executes control for changing sound currently outputted by the speaker to different sound.

17. The control device according to claim 15, wherein when the output device is an air conditioner, the controlling circuitry executes at least one of control for changing a direction of air currently outputted by the air conditioner to a different direction and control for changing wind power of the air conditioner.

18. The control device according to claim 15, wherein when the output device is a smell generator, the controlling circuitry executes control for changing a smell currently outputted by the smell generator to a different smell.

19. The control device according to claim 15, wherein when the output device is an illuminator, the controlling circuitry executes control for changing illuminance, illumination color or an illumination angle of light currently outputted by the illuminator.

20. A control system comprising:
an image capturing device; and
a control device for controlling an output device that outputs a stimulus,
wherein the control device includes:
acquiring circuitry to acquire a video obtained by the image capturing device by capturing images of a plurality of people existed in a place where movement in a plurality of directions is possible; and
controlling circuitry to detect a moving people count as a number of people who moved in a first direction among the plurality of directions based on the video, calculate a ratio of the people who moved in the first direction by using the moving people count, and when a situation where the ratio is less than a predetermined threshold value is continuing for a predetermined set time, execute control for changing the stimulus currently outputted by the output device to a different stimulus.

21. A control system comprising:
a first image capturing device that is installed at a first spot and transmits an image or a video obtained by capturing one or more images of a plurality of people;
a first output device that is installed at the first spot;
a second output device that is installed at a second spot as a spot after the first spot;
a control device that manages information to be outputted by the first output device; and
a second image capturing device that is installed at the second spot and transmits an image or a video obtained by capturing one or more images of a plurality of people,
wherein the control device
calculates a first visual recognition rate regarding information currently outputted by the first output device based on the image or the video received from the first image capturing device,
calculates a second visual recognition rate regarding information currently outputted by the second output device based on the image or the video received from the second image capturing device, and
when the first visual recognition rate and the second visual recognition rate are lower than a predetermined threshold value, executes control for making the second output device output information different from the information currently outputted by the first output device.

22. A control system comprising:
a first image capturing device that is installed at a first spot and transmits an image or a video obtained by capturing one or more images of a plurality of people;
a first output device that is installed at the first spot;
a first control device that manages information to be outputted by the first output device;
a second output device that is installed at a second spot as a spot after the first spot; and
a second control device, wherein
the first control device calculates a first visual recognition rate regarding information currently outputted by the first output device based on the image or the video received from the first image capturing device, and when the first visual recognition rate is lower than a predetermined threshold value, notifies the second control device that the first visual recognition rate is lower than the threshold value,
when the first visual recognition rate is lower than the threshold value, the second control device transmits a transmission request, requesting transmission of the information currently outputted by the first output device, to the first control device,
the first control device receiving the transmission request transmits the information currently outputted by the first output device to the second control device, and
the second control device receiving the information currently outputted by the first output device executes control for making the second output device output information different from the information currently outputted by the first output device.

23. The control system according to claim 22, further comprising a second image capturing device that is installed at the second spot,
wherein the second control device
calculates a second visual recognition rate regarding information currently outputted by the second output device based on an image or a video received from the second image capturing device, and
when the first visual recognition rate and the second visual recognition rate are lower than the threshold value, transmits the transmission request to the first control device.

24. A control method performed by a control device for controlling an output device that outputs a stimulus, the control method comprising:
acquiring a video obtained by an image capturing device by capturing images of a plurality of people existed in a place where movement in a plurality of directions is possible;
detecting a moving people count as a number of people who moved in a first direction among the plurality of directions based on the video;
calculating a ratio of the people who moved in the first direction by using the moving people count; and
when a situation where the ratio is less than a predetermined threshold value is continuing for a predetermined set time, executing control for changing the stimulus currently outputted by the output device to a different stimulus.

25. A control method performed by a control device for communicating with a display device that displays information and controlling an output device that outputs a stimulus, the control method comprising:
acquiring an image or a video obtained by an image capturing device by capturing one or more images of a plurality of people;
calculating a visual recognition rate, as a ratio at which the information displayed by the display device is visually recognized thanks to the stimulus outputted by the output device, based on the image or the video; and
when the visual recognition rate is lower than a predetermined threshold value, executing control for changing the stimulus currently outputted by the output device to a different stimulus without changing the information displayed by the display device.

26. A control device for controlling an output device that outputs a stimulus, the control device comprising:
a processor to execute a program; and
a memory to store the program which, when executed by the processor, performs processes of,
acquiring a video obtained by an image capturing device by capturing images of a plurality of people existed in a place where movement in a plurality of directions is possible,
detecting a moving people count as a number of people who moved in a first direction among the plurality of directions based on the video,
calculating a ratio of the people who moved in the first direction by using the moving people count, and
when a situation where the ratio is less than a predetermined threshold value is continuing for a predetermined set time, executing control for changing the stimulus currently outputted by the output device to a different stimulus.

27. A control device for communicating with a display device that displays information and controlling an output device that outputs a stimulus, the control device comprising:
a processor to execute a program; and
a memory to store the program which, when executed by the processor, performs processes of,
acquiring an image or a video obtained by an image capturing device by capturing one or more images of a plurality of people,
calculating a visual recognition rate, as a ratio at which the information displayed by the display device is visually recognized thanks to the stimulus outputted by the output device, based on the image or the video, and
when the visual recognition rate is lower than a predetermined threshold value, executing control for changing the stimulus currently outputted by the output device to a different stimulus without changing the information displayed by the display device.
